(19) United States
(12) Patent Application Publication
(10) Pub. No.: US 2024/0385159 A1
(43) Pub. Date: Nov. 21, 2024

(54) NEURAL NETWORK-ENHANCED CONTAMINANT MEASUREMENTS

(71) Applicant: ECOMESURE, Saclay (FR)

(72) Inventors: Damien PELLETIER, Orsay (FR); Fabio FURLAN, Saint-Michel-Sur-Orge (FR); Thomas BROUSSARD, Le Plessis-Pate (FR); Aymane SOUANI, Evry (FR)

(21) Appl. No.: 18/693,069

(22) PCT Filed: Oct. 11, 2022
(86) PCT No.: PCT/EP2022/078306

§ 371 Date: Mar. 18, 2024

(30) Foreign Application Priority Data
Oct. 15, 2021 (FR) 2110983

(51) Int. Cl. G01N 33/00

(52) U.S. Cl. CPC G01N 33/0006; G01N 33/0016

(57) ABSTRACT

A method for enhancing measurements from a contaminant measurement sensor, the measurement sensor being connected to a specialized remote server including a neural network, the method including the following steps: creating a trained neural network model based on the neural network, and for each measurement performed by the measurement sensor: measuring at least one raw data item by means of the measurement sensor, sending the at least one raw data item to the specialized remote server, and enhancing the at least one raw data item by means of the trained neural network model, making it possible to obtain the measurement of the concentration of the contaminant.

### TECHNICAL FIELD

The present invention relates to a method for enhancing measurements from a contaminant measurement sensor.

### State of the Prior Art

Since COP21 it has been possible to observe a rapid expansion of systems for measuring atmospheric contaminants which, in combination with, for example, crowdsourcing, make a spatial representation of air quality possible. This mapping, on the initiative of the traditional players in monitoring for local bodies, is in its infancy.

Nevertheless, it raises the question of uncertainty with regard to the data items, the use thereof and the possibilities offered by new technologies stemming from artificial intelligence.

It also raises the issue of the drift of the measurement sensors between calibration in the laboratory and their use in the field. Ultimately, the accuracy of these sensors is relative and the measurement uncertainties are still poorly known.

Linear calibrations of contaminant measurement sensors in the laboratory are known from the state of the art. In fact, there are linear models for conventional two-point calibration making it possible to obtain good results. A first measurement of the zero point of the sensor is performed by applying a zero concentration of gas or particles, then a second measurement of the operating point of the sensor is performed by applying a known concentration. On the basis of the measurements of the zero point and of the known concentration point, the equation of the calibration line is obtained, making it possible to determine the concentrations as a function of the measurements from the sensor. As this model is linear, it is hypothesized that the operating curve of the sensor is too, although this is not the case.

In addition, a calibration carried out in the laboratory makes it possible to set the measurements at the beginning, but a drift of these latter is noted over time. The laboratory conditions only partially represent the real conditions in the field. Such a model does not make it possible to compensate for the non-linear drifts noted on site after the sensor leaves the laboratory or after several months of use thereof.

In addition, such a linear model does not make it possible to take into account cross sensitivities between the contaminants, as the sensors which are sensitive to a main gas are, for example, also capable of being sensitive to other gases. For example, the SO2 sensor is sensitive to NO2, which distorts the SO2 measurement if NO2 is present.

The aim of the present invention is to resolve at least one of these problems by means of a new method for enhancing measurements from a contaminant measurement sensor. One of the aims of the present invention is thus to improve the accuracy of the measurements from the contaminant measurement sensor.

Another aim of the present invention is to improve the calibration of the contaminant measurement sensor.

### DISCLOSURE OF THE INVENTION

This objective is achieved with a method for enhancing measurements from a contaminant measurement sensor, the measurement sensor being connected to a specialized remote server comprising a neural network, the method comprising the following steps:
creating a trained neural network model based on the neural network, and
   for each measurement performed by the measurement sensor:
      measuring at least one raw data item by means of the measurement sensor,
      sending the at least one raw data item to the specialized remote server,
      enhancing the at least one raw data item by means of the trained neural network model, making it possible to obtain the measurement of the concentration of the contaminant.

The contaminant can be, for example, a gas or a particle.

By “enhancing a raw data item” is meant an adjustment of the value of the raw data item after application of the trained neural network model.

The method according to the invention consists of modelling the behaviour of the measurement of the contaminants by means of a neural network adapted so as to make a non-linear calibration of the measurement sensor possible.

The method makes it possible to cause the measurement accuracy of the contaminant sensors to tend towards the reference and also makes an improvement of the chemical and electronic accuracy of the measurements possible.

The method according to the invention makes it possible to improve the accuracy of the measurements using artificial intelligence technologies such as neural networks. The neural network model of the invention is adapted to the field of the measurement of contaminants, whether gaseous or particulate.

Advantageously, the step of creating a trained neural network model can be broken down into three sub-steps:
generating a scenario for calibrating the measurement sensor, the calibration scenario being generated based on an instruction for measuring parameters as a function of time, said parameters comprising at least one environmental parameter,
creating a neural network model by supplying the neural network with the calibration scenario of said measurement sensor,
training the neural network model by varying the at least one environmental parameter.

The training of the neural network model according to the invention makes it possible to take into account the distinctive nature of the location where the sensor is used, such as for example an urban environment, by the side of a ring road, a motorway, a country location or also an industrial environment. This thus makes it possible to adjust the measurements taking into account the environment in which the sensor is placed. Each measurement sensor has its own neural network model with its own parameters originating from its training. The neural network model can be archived on the specialized remote server.

The measurement sensor can be calibrated a first time based on the calibration scenario, the first calibration corresponding to a multipoint calibration. The first calibration of the sensor is non-linear. It makes it possible to improve its accuracy and to establish a first neural network model by supplying the neural network with its calibration scenario.

The measurement sensor can be associated to a reference apparatus, the measurement sensor being calibrated a second time after a use period T with respect to the reference apparatus. Due to the non-linearity, the method makes it possible to compensate for the non-linear drifts noted on site after the measurement sensor leaves the laboratory or after several months of use. The method according to the invention then makes it possible to improve the measurements from the measurement sensor by means of the calibration thereof.

The measurement instruction can comprise at least ten levels starting from a zero concentration to a concentration corresponding to the maximum of the measurement sensor.

Advantageously, for each level of the measurement instruction, at least one of the measurements of the following parameters can be recorded automatically:
concentration of the instruction,
concentration of a reference apparatus,
measured raw concentration of the contaminant,
measured concentration of at least one other contaminant which can act in terms of cross sensitivity,
a temperature,
a relative humidity,
a pressure.

The method thus takes account of cross sensitivities between contaminants, for example: SO2 with NO2, CO with CO2, NO2 with O3.

The second calibration of the measurement sensor can generate new parameter measurements, the new parameter measurements being supplied to the neural network to update the trained neural network model.

A second trained neural network model can be created based on the updating of the trained neural network model, the final trained neural network model of the measurement sensor corresponding to the product of the two trained neural network models. The final enhanced measurement is thus the result of the cumulative application (analogy with a product of neural matrices) of the N trained neural network models. In this way, a new neural network model does not destroy the previous one, but acts as a complement for adjusting the previous neural network model. This method is similar to transfer learning and makes a learning resulting from several previous learning processes possible.

The step of creating the trained neural network model can be performed in the laboratory. The first calibration of the measurement sensor can also be performed in the laboratory.

By way of non-limitative example and in addition to the above, the neural network model can be obtained by:
applying an algorithm with multiple layers and inputs taking into account the physical attributes of the sensor, alternately of the “classification” and “regression” type, to the raw data item measured;
taking into account the “lags”, i.e. previous measurement data items;
using “sigmoid” activation functions; and
integrating additional input data items, such as for example temperature T, pressure P, relative humidity RH, measurements of other contaminants etc.

The second calibration of the measurement sensor can be performed remotely. In fact, a non-linear calibration as presented by the invention makes it possible to perform the calibration of the measurement sensor remotely and to improve the measurements if they drift.

The measurement of the concentration of the contaminant can be obtained in real time.

### DESCRIPTION OF THE FIGURES AND EMBODIMENTS

Other advantages and characteristics of the invention will become apparent on reading the detailed description of implementations and embodiments which are in no way limitative, and from the following attached drawings:

FIG. 1 shows a system for measuring a contaminant by means of a measurement sensor according to an embodiment of the invention,

FIG. 2 shows the general principle of the method of the present invention,

FIG. 3 shows a diagram for generating a calibration instruction according to an embodiment of the present invention,

FIG. 4 shows an example of measurements of CO2 gas that are not enhanced by a neural network according to the present invention,

FIG. 5 shows a graph of the results of the enhanced measurement of CO2 gas in FIG. 4,

FIG. 6 is a graph showing the phenomenon of cross sensitivities between two contaminants.

As these embodiments are in no way limitative, variants of the invention can in particular be considered comprising only a selection of the characteristics described or illustrated hereinafter, in isolation from the other characteristics described or illustrated, (even if this selection is isolated within a sentence comprising these other characteristics), if this selection of characteristics is sufficient to confer a technical advantage or to differentiate the invention with respect to the state of the prior art. This selection comprises at least one, preferably functional, characteristic without structural details, and/or with only a part of the structural details if this part alone is sufficient to confer a technical advantage or to differentiate the invention with respect to the state of the prior art.

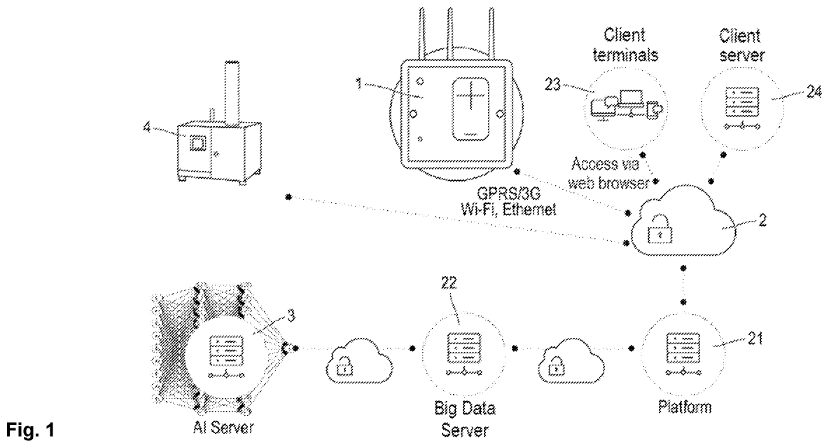

With reference to FIG. 1, a system for measuring a contaminant by means of a sensor according to an embodiment of the invention will be described first. The system comprises a contaminant measurement sensor 1, a network 2 of the internet type, a specialized remote server 3 and a reference apparatus 4. The measurement sensor 1, the specialized remote server 3 as well as the reference apparatus 4 are connected via the network 2. The specialized remote server 3 comprises a neural network (not shown in the figure). A platform 21, accessible to the client terminals 23 and the client server 24 via the network 2, is associated to a big data server 22 having more than 100 billion environmental data items.

The method of the present invention is associated with the measurement sensor 1.

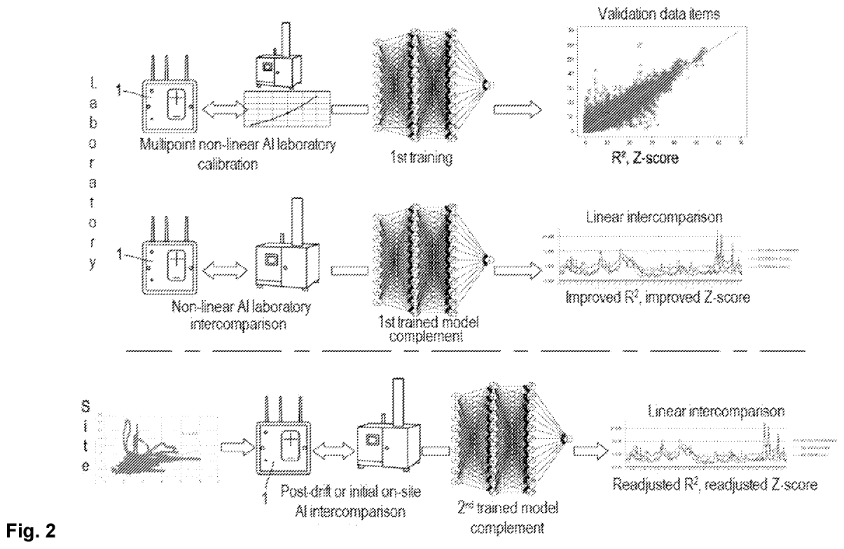

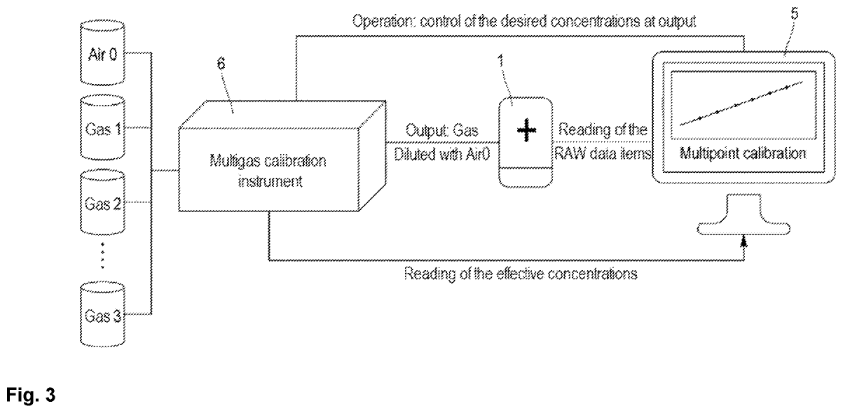

According to FIG. 2, which shows the general principle of the method, the measurement sensor 1 is calibrated a first time in a laboratory. The measurement sensor 1 is placed in a calibration chamber having a controlled concentration in the laboratory on a test bed the operation of which is shown in FIG. 3.

The test bed comprises a multigas calibration instrument 6 and a computer 5. The computer 5 makes it possible to control the concentrations of gas at output from the measurement sensor 1 and also makes the reading of the effective concentrations possible. Each gas is sent to the calibration instrument 6 at a precise concentration level defined by the computer 5. The multigas calibration instrument 6 then transfers it to the measurement sensor 1. The different gases are for example: CO, CO2, NO, NO2, O3, NH3, H2S. The gas sent is diluted with air 0. Reference instruments are used to measure the concentrations of gas which constitute the target values for the calibration.

The test bed then makes it possible to generate a calibration scenario. The calibration scenario corresponds to a measurement instruction for concentrations or parameters as a function of time. The measurement instruction is initiated by the computer 5, for example, ten levels starting from a zero concentration to a concentration corresponding to the maximum of the measurement sensor 1 as a function of time. The number of levels can be comprised between 10 and 100. For each level, at least one of the following concentrations is automatically recorded by the bed:
Concentration of the instruction,
Concentration of the reference apparatus,
Raw concentration of the gas measured by the sensor,
Concentration of the other gases which can act in terms of cross sensitivity.

For each level, at least one of the following parameters is also automatically recorded by the bed: measurement of temperature, relative humidity and pressure. Other concentrations or environmental parameters can also be recorded.

The different measurements of concentrations and parameters make it possible to calibrate the sensor a first time. It is a multipoint calibration.

When the measurement sensor is calibrated, the method sends the calibration scenario which is constituted by the matrix of previous measurements, to the specialized remote server 3 so as to feed the neural network. A neural network model is created. This “fed” network constitutes the laboratory calibration matrix of the sensor.

The method then initiates a learning of the neural network model by varying certain parameters which are considered stable in the calibration chamber such as for example the environmental parameters (T, P, RH). The variations of the parameters bring new data items which serve to feed the neural network once more, which updates the neural network model. The neural network model then corresponds to a trained neural network model (1st learning complement). A second neural network model is created, this second neural network model corresponding to the first neural network model with the first learning complement. The final trained neural network model corresponds to the product of the first neural network model and the second neural network model. The final enhanced measurement of the measurement sensor 1 is thus the result of the cumulative application (analogy with a product of neural matrices) of the N trained models. In this way, a new model does not destroy the previous one, but acts as a complement for adjusting the previous model.

The measurement sensor 1 is then deployed on the client's site. According to FIG. 1, the measurement sensor 1 is connected to a reference apparatus 4 via the network 2. The measurement sensor 1 is connected to the network 2 via a wireless connection. The measurement sensor 1 performs measurements of the ambient air on the client site. The measurements performed are sent in the form of raw data items to the specialized remote server 3 via the network 2 and the platform 21. The big data server 22 stores all of the raw measurements performed by the measurement sensor 1.

Once the specialized remote server 3 has received the raw data items, these are processed and sent to the final trained neural network model which will enhance said measurements performed by the measurement sensor 1 so as to provide the measured concentration of the contaminant. The enhanced measurements are available in real time on the platform 21 via the network 2.

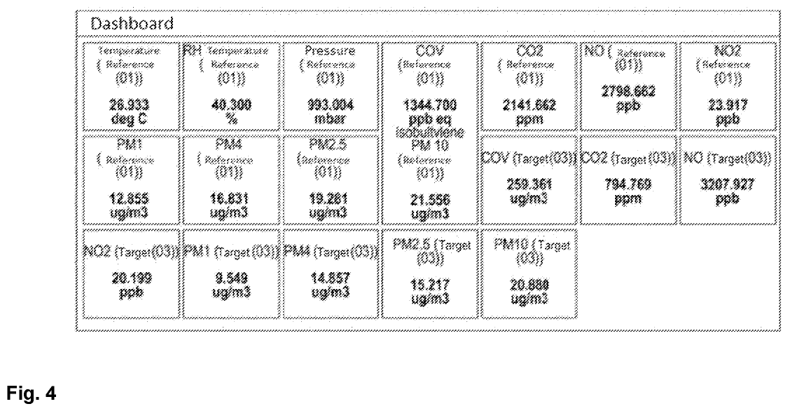

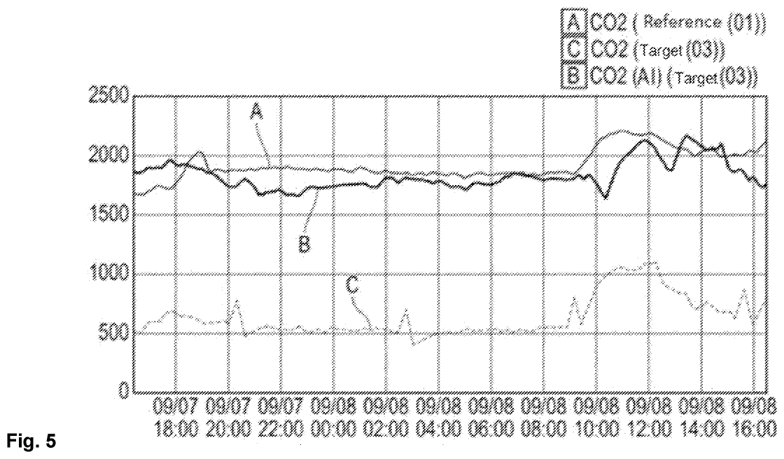

FIG. 4 shows an example of real time measurements of contaminants of the reference apparatus and target sensors. FIG. 5 shows the measurement curves of the CO2 gas sensor enhanced by means of the method of the present invention. The measurements from the measurement sensor 1 are referenced on the platform 21. FIG. 5 comprises three curves, curve A, B and C. Curve A represents the reference curve, curve B represents the target curve obtained according to the present invention by applying the neural network, and curve C represents the target curve which is calibrated linearly. It is noted that curve B is very close to reference curve A, while curve C remains very far from the reference curve.

The measurement sensor 1 is once again calibrated after a period of time T of use or when a drift in its measurements is noted. The period of time is comprised between 2 months and 2 years for example.

The measurement sensor 1 is recalibrated with respect to the reference machine 4, the sensor being located at a contained distance, several tens of metres for example, from said machine 4. When the measurement sensor 1 is recalibrated, an improvement is noted in the measurements carried out by the measurement sensor 1. This second calibration makes it possible to avoid the drift of the measurements performed by the measurement sensor 1 over time.

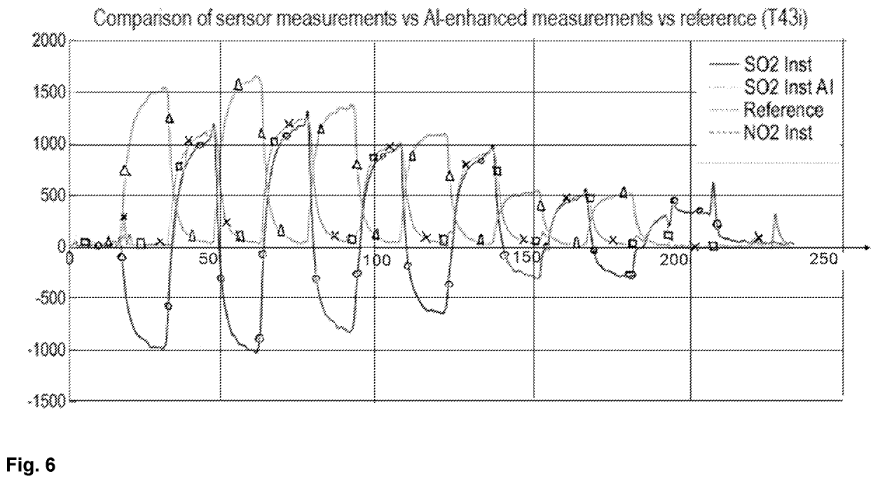

The present invention advantageously makes it possible to take into account the sensitivity of a contaminant with respect to another, i.e. one contaminant interfering with or disrupting another. In general, it is a mutual disruption. In FIG. 6, four measurement curves are distinguished. The x-axis represents a time scale. The y-axis represents the concentration of the contaminant in ppb, ppm or μg/m3.

The curve with triangles represents the NO2 concentration.

The curve with squares represents the SO2 concentration measured by the reference apparatus.

The curve with circles represents the SO2 concentration measured using the linear method, i.e. without enhancement according to the present invention.

The curve with crosses represents the SO2 concentration measured with enhancement according to the present invention.

The curve according to the linear method is close to the curve for measurement by the reference apparatus in the absence of NO2, but becomes negative as soon as NO2 is present. This demonstrates the influence of NO2 on the detection of SO2.

The measurement according to the invention makes it possible to take into account the sensitivity of the SO2 to NO2 and to obtain an enhanced measurement very close to the reference. In fact, the curve with crosses follows the reference measurement curve by eliminating the influence of NO2 as far as possible.

Typically, at least one of the means of the device according to the invention described above, preferably each of the means of the device according to the invention described above are technical means.

Typically, each of the means of the device according to the invention described above can comprise at least one computer, a central processing or calculation unit, an analogue electronic circuit (preferably dedicated), a digital electronic circuit (preferably dedicated), and/or a microprocessor (preferably dedicated) and/or software means.

Of course, the invention is not limited to the examples which have just been described, and numerous adjustments may be made to these examples without departing from the scope of the invention.

Of course, the different characteristics, forms, variants and embodiments of the invention can be combined together in various combinations inasmuch as they are not incompatible or mutually exclusive. In particular, all the variants and embodiments described above can be combined together.

1. A method for enhancing measurements from a contaminant measurement sensor, the measurement sensor being connected to a specialized remote server comprising a neural network, the method comprising the following steps:
creating a trained neural network model based on the neural network; and
for each measurement performed by the measurement sensor:
measuring at least one raw data item by means of the measurement sensor;
sending the at least one raw data item to the specialized remote server; and
enhancing the at least one raw data item by means of the trained neural network model, making it possible to obtain the measurement of the concentration of the contaminant.

2. The method according to claim 1, characterized in that the step of creating a trained neural network model is broken down into three sub-steps:
generating a scenario for calibrating the measurement sensor, the calibration scenario being generated from a parameter measurement instruction as a function of time, said parameters comprising at least one environmental parameter;
creating a neural network model by supplying the neural network with the calibration scenario of said measurement sensor; and
training the neural network model by varying the at least one environmental parameter.

3. The method according to claim 2, characterized in that the measurement sensor is calibrated a first time based on the calibration scenario, the first calibration corresponding to a multipoint calibration.

4. The method according to claim 3, characterized in that the measurement sensor is associated to a reference apparatus, the measurement sensor being calibrated a second time after a use period T with respect to the reference apparatus.

5. The method according to claim 2, characterized in that the measurement instruction comprises at least ten levels starting from a zero concentration to a concentration corresponding to the maximum of the measurement sensor.

6. The method according to claim 5, characterized in that, for each level of the measurement instruction, at least one of the measurements of the following parameters is recorded automatically by the measurement sensor:
concentration of the instruction,
concentration of a reference apparatus,
measured raw concentration of the contaminant,
measured raw concentration of at least one other contaminant which can act in terms of cross sensitivity,
a temperature,
a relative humidity,
a pressure.

7. The method according to claim 4, characterized in that the second calibration of the measurement sensor generates new parameter measurements, the new parameter measurements being supplied to the neural network to update the trained neural network model.

8. The method according to claim 7, characterized in that a second trained neural network model is created based on the update of the trained neural network model, the final trained neural network model of the measurement sensor corresponding to the product of the two trained neural network models.

9. The method according to claim 4, characterized in that the second calibration of the measurement sensor is carried out remotely.

10. The method according to claim 1, characterized in that the measurement of the concentration of the contaminant is obtained in real time.
